(19) United States
(12) Patent Application Publication
(10) Pub. No.: US 2015/0293117 A1
(43) Pub. Date: Oct. 15, 2015

(54) DETECTION DEVICE, DETECTION METHOD AND DETECTION STRIP

(71) Applicant: National Tsing Hua University, Hsinchu city (TW)

(72) Inventors: Chao-Kai HSU, Hsinchu city (TW); Hsin-Yu HUANG, Hsinchu city (TW); Hiroshi SHIMIZU, Hsinchu city (TW); Chao-Min CHENG, Hsinchu city (TW)

(21) Appl. No.: 14/516,098

(22) Filed: Oct. 16, 2014

(30) Foreign Application Priority Data
Apr. 15, 2014 (TW) 103113744

(51) Int. Cl. G01N 33/68

(52) U.S. Cl. CPC G01N 33/6854; G01N 2800/20

(57) ABSTRACT

A detection device applied for detecting body fluids includes a substrate and a plurality of antigens of type XVII collagen. The substrate includes at least one reaction portion. The reaction portion includes a fiber-based material. Antigens of type XVII collagen are disposed on the fiber-based material. The present invention further provides a detection method and a detection strip for detecting body fluids. The present invention is advantageous for easy operation, lower amount of reagents and rapid analysis.

### CROSS REFERENCE TO RELATED APPLICATIONS

This Non-provisional application claims priority under 35 U.S.C. §119(a) on Patent Application No(s). 103113744 filed in Taiwan, Republic of China on Apr. 15, 2014, the entire contents of which are hereby incorporated by reference.

### BACKGROUND OF THE INVENTION

1. Field of Invention

The present invention relates to a detection device, a detection method, and a detection strip, and more particularly, to a detection device, a detection method, and a detection strip applied for detecting body fluid.

2. Related Art

Bullous pemphigoid is a common systemic autoimmune blistering disease frequently occurring in senior persons. It is characterized by generalized skin blistering and relapse. About two-thirds of cases occur in the elderly persons. Bullous pemphigoid is a kind of chronic disease. Clinically, patients with BP show multiple features, such as tense blisters, erosions and crusts with itchy urticarial plaques. Bullous pemphigoid causes high mortality (19˜26%) and costs high medical care spending. This disease results from the autoantibodies against type XVII collagen (so called COL17 or BP180 and BPAG2). The most important epitope in type XVII collagen is located at the extracellular portion of the 16th non-collagenous domain, termed NC16A.

In recent years, the clinical diagnosis of bullous pemphigoid primarily focuses on clinical pathological features or relies on tissue sections and immunofluorescence studies. Detection of autoantibodies targeting the NC16A domain of type XVII collagen using enzyme-linked immunosorbent assay (ELISA) has demonstrated successful application and efficacy for diagnosing BP, which is advantageous for assisting clinicians to decide the dosage of Immunosuppressive drugs.

However, the detection of traditional method needs to obtain detection sample by drawing blood of patients. Otherwise, relatively large number of reagents is required, and longer time is required for detection, which virtually extending the time for diagnosing bullous pemphigoid.

Therefore, it is an important subject to provide a detection device capable of decreasing the needed sample amount and obtaining the advantages of easy and rapid operation in order to improve clinical applicability.

### SUMMARY OF THE INVENTION

In view of the foregoing, it is an object of the present invention to provide a detection device capable of decreasing the needed sample amount and obtaining the advantages of easy and rapid operation in order to improve clinical applicability.

To achieve the above, the present invention discloses a detection device applied for detecting body fluids comprising a substrate and a plurality of antigens of type XVII collagen. The substrate includes at least one reaction portion with a fiber-based material. Antigens of type XVII collagen are disposed on the fiber-based material.

In one embodiment of the present invention, the body fluid sample is a superficial body fluid sample.

In one embodiment of the present invention, the detection device is a bullous pemphigoid detection device.

In one embodiment of the present invention, the substrate includes at least one non-reaction portion, and the non-reaction portion is covered by hydrophobic material.

In one embodiment of the present invention, the substrate includes two reaction portions, and the reaction portions are separated by the non-reaction portion.

In one embodiment of the present invention, the fiber-based material is high density fiber-based material with an average pore size ranged from 0.7 to 12 micrometers.

To achieve the above, the present invention discloses a detection method applied for detecting a superficial body fluid sample of an organism comprising the following steps: providing a detection device comprising a substrate including at least one reaction portion with a fiber-based material and a plurality of antigens of type XVII collagen disposed on the fiber-based material; contacting the detection device with an affected area of the organism and making the superficial body fluid sample of the organism attach the reaction portion of the detection device; and detecting the interactive reaction of the body fluid sample and the antigens of type XVII collagen.

In one embodiment of the present invention, the detection method is applied for detecting bullous pemphigoid.

In one embodiment of the present invention, the superficial body fluid sample includes an antibody or an antibody fragment capable of specifically recognizing the antigens of type XVII collagen.

To achieve the above, the present invention discloses a detection strip applied for detecting a body fluid sample comprising a sampling portion, a transferring portion, and a reaction portion. The transferring portion is disposed between the sampling portion and the reaction portion. The reaction portion includes a fiber-based material. A plurality of antigens of type XVII collagen are disposed on the fiber-based material.

In one embodiment of the present invention, the body fluid sample is a superficial body fluid sample.

In one embodiment of the present invention, the detection strip is a bullous pemphigoid detection device.

In one embodiment of the present invention, the fiber-based material is high density fiber-based material with an average pore size ranged from 0.7 to 12 micrometers.

In one embodiment of the present invention, the detection strip is applied with a monitoring device, and the monitoring device detects the interactive reaction of the body fluid sample and the antigens of type XVII collagen.

As mentioned above, the detection device, detection method and the detection strip of the present invention is applied with a reaction portion including fiber-based material and antigens of type XVII collagen disposed thereon. The detection, detection method and the detection strip device detect the antibody included in the superficial body fluid of bullous pemphigoid patients, especially the antibody capable of specifically recognizing type XVII collagen or NC16A domain of type XVII collagen. Since the detection device, detection method and the detection strip of the present invention merely need to absorb the superficial body fluid with simple contacting or pasting onto the affected area of the patients, the time for tradition invasive sampling with blood drawing and additional operating may be saved. Preferably, the techniques using blood as the detection target which needs to undergone the additional processes like preprocess and separation may lost certain amount of detection sample. Thus, larger amount of detection sample must be obtained in order to achieve accurate result. On the contrary, the present invention disposes the superficial body fluid on the detection device without extra process, which is advantageous for small amount of sample.

### BRIEF DESCRIPTION OF THE DRAWINGS

The present invention will become more fully understood from the subsequent detailed description and accompanying drawings, which are given by way of illustration only, and thus are not limitative of the present invention and wherein:

FIG. 1A shows schematic view of a detection device according to preferred embodiment of the present invention.

FIG. 1B shows partially schematic view of the region A of the detection device according to FIG. 1A.

FIG. 1C shows schematic view of section line A-A shown in FIG. 1A.

FIG. 2A shows schematic view of a detection device according to another preferred embodiment of the present invention.

FIG. 2B shows schematic view of section line a-a shown in FIG. 2A.

FIG. 3 shows schematic view of a detection device according to another preferred embodiment of the present invention.

FIG. 4 is a flow chart showing the steps of the detection method for bullous pemphigoid according to preferred embodiment of the present invention.

FIG. 5 shows schematic view of detection strip according to preferred embodiment of the present invention.

FIG. 6 shows the result of the intensity ratio of monoclonal antibody for anti-NC16A by paper-based detection device applying ELISA system.

FIG. 7 shows the result of the relative intensity of autoantibody produced by patients for anti-NC16A by paper-based detection device applying ELISA system.

FIG. 8 shows the result of the relative intensity of antibody collected from patients' serum for anti-NC16A by paper-based detection device applying ELISA system.

FIG. 9 shows the result of the relative intensity of antibody collected from patients' superficial body fluid for anti-NC16A by paper-based detection device applying ELISA system.

### DETAILED DESCRIPTION OF THE INVENTION

The present invention will be apparent from the following detailed description, which proceeds with reference to the accompanying drawings, wherein the same references relate to the same elements.

The detection device of the present invention is applied with Enzyme-linked immunosorbent assay (ELISA). The analyte of the present invention is obtained from the body fluid sample of an organism, preferably from the superficial body fluid sample of the organism. The word “body fluid” used here is collectively referred to the extracellular fluid exuding from the complete superficial tissue of the organism, or the open holes caused by disease or trauma.

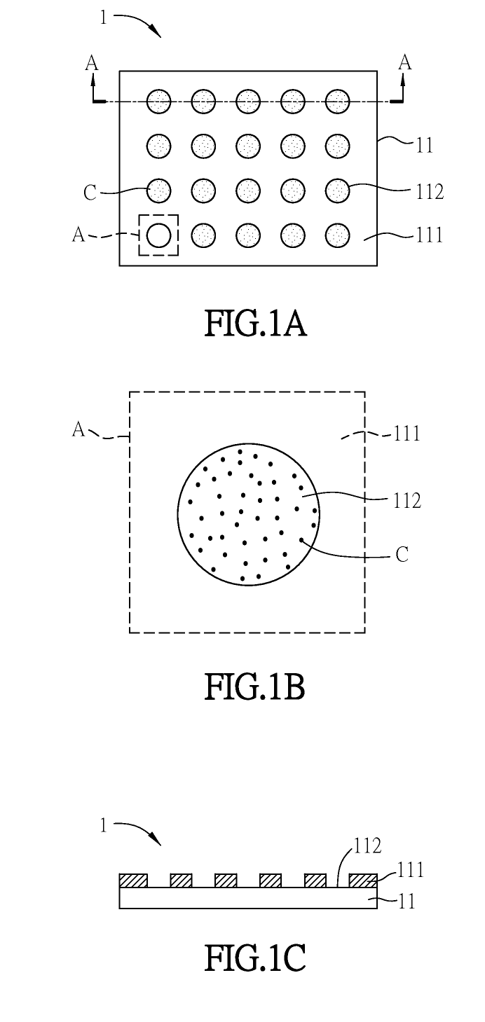

FIG. 1A shows schematic view of a detection device according to preferred embodiment of the present invention, FIG. 1B shows partially schematic view of the region A of the detection device according to FIG. 1A, and FIG. 1C shows schematic view of section line A-A shown in FIG.1A. With reference to FIG. 1A˜1C, the detection device 1 includes a substrate 11 and a plurality of antigens of type XVII collagen C. The substrate 11 includes at least one non-reaction portion 111 and at least one reaction portion 112. This embodiment is based on a substrate 11 having a non-reaction portion 111 as an example. The non-reaction portion 111 defines the plural reaction portions 112. That is, the reaction portions 112 are separated by the non-reaction portion 111. However, the number, shape, size of the reaction portion 112 is not limitation of the present invention. It can be designed according to the need of the experiment.

With reference to FIG. 1A and FIG. 1B, in this embodiment, the non-reaction portion 111 of the substrate 11 is covered by hydrophobic material. Thus, the non-reaction portion 111 is also called hydrophobic region. In detail, the method of forming the hydrophobic non-reaction portion 111 includes wax printing. Wax printing applies device with wax spray function, such as a printer. Wax is disposed on the substrate 11 according to the user's option of figure, shape, or size. With the hydrophobic non-reaction portion 111, the body fluid sample is restricted in the reaction portion 112 in order to prevent the loss of the sample and improve the level of detection accuracy.

However, the formation method of the non-reaction portion 111 is not limitation of the present invention. In practical use, the hydrophilic substrate 11 may alternatively be coated with photoresist layer. Specifically speaking, when the negative resist is applied, such as SU-8 epoxy-based negative photoresist, the region irradiated by UV light would not dissolve in photoresist developer. Thus, the non-reaction portions 111 are formed. On the contrary, the regions which are not irradiated by UV light form the hydrophilic reaction portion 112. However, the detailed forming method is well-understood by the person having ordinary skill in the art, and is not repeated here.

The substrate 11 applies fiber-based material, such as botanical fiber 1 as its material; preferably the material is a high-density fiber. In this embodiment, the high density fiber-based material with an average pore size ranged from 0.7 to 12 micrometers, preferably ranged from 1 to 10 micrometers. The practical application range and preferable application range both includes the combination of any two integers in the above mentioned range. While choosing substrate with larger pore size, the body fluid sample may affected by the action of siphon force and capillary force, thus providing faster flow rates; on the contrary, while choosing substrate with smaller pore size, the flow rate of the body fluid sample may become relatively slower and due to the action of siphon force. On the detection effect side, higher flow rate may not be advantageous for the detection target immobilizing in the reaction portion 112; on the contrary, more non-specific binding may occurred with lower flow rate. Therefore, in practical use, the average pore size of high density fiber-based material is selected according to the component included in the body fluid sample, thus being adjusted by the practical use and need.

In this embodiment, the reaction portion 112 is the region surrounded and defined by the hydrophobic non-reaction portion 111 (i.e. partial region of substrate 11). Thus, the reaction portion 112 also includes the hydrophilic ability the same as the fiber-based material. The reaction portion 112 maintains and absorbs the body fluid by the capillary action generated by the fiber-based material. Furthermore, the superficial body fluid is able to be wicked, diffusing and transferring in the reaction portion due to the density and the minor groove of the fiber-based material. Compared to the nitrocellulose paper applied in the prior technique used for absorbing the sample merely on the surface, the high density fiber-based material of the present invention includes better water permeability, thus effectively improving the subsequent detection accuracy of the ELISA analysis.

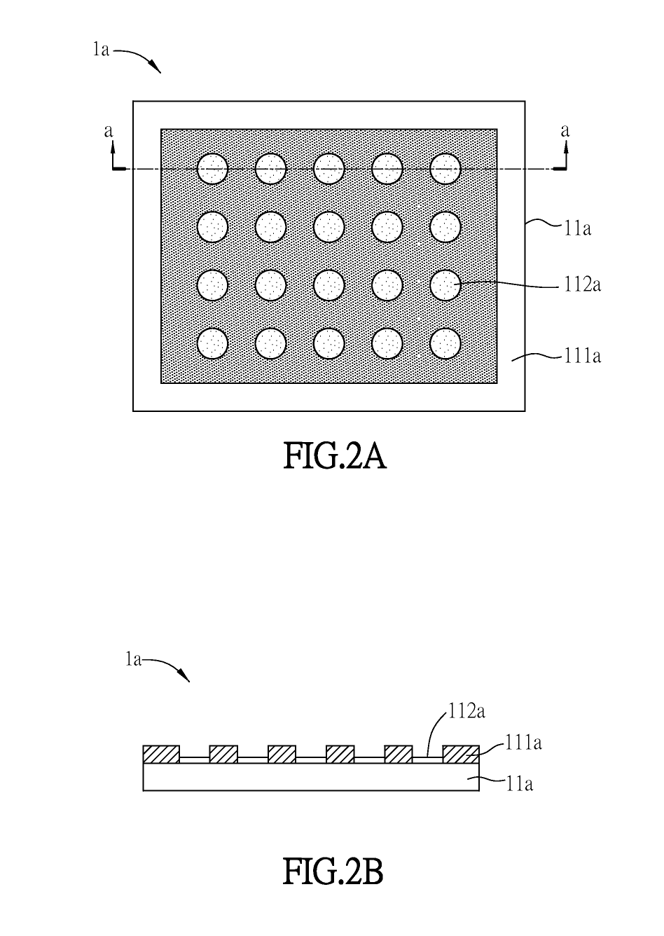

In other embodiments, the reaction portion may be disposed by additionally adding fiber-based material on the substrate. With reference to FIG. 2A and FIG. 2B, the same as the above-mentioned embodiment, the reaction portion 112a of the detection device 1a is defined by the non-reaction portion 111a through wax printing. The reaction portion is additionally disposed with fiber-based material in order to play the detection region for superficial body fluid detection. And the material of the substrate 11a is not limited to high density fiber-based material.

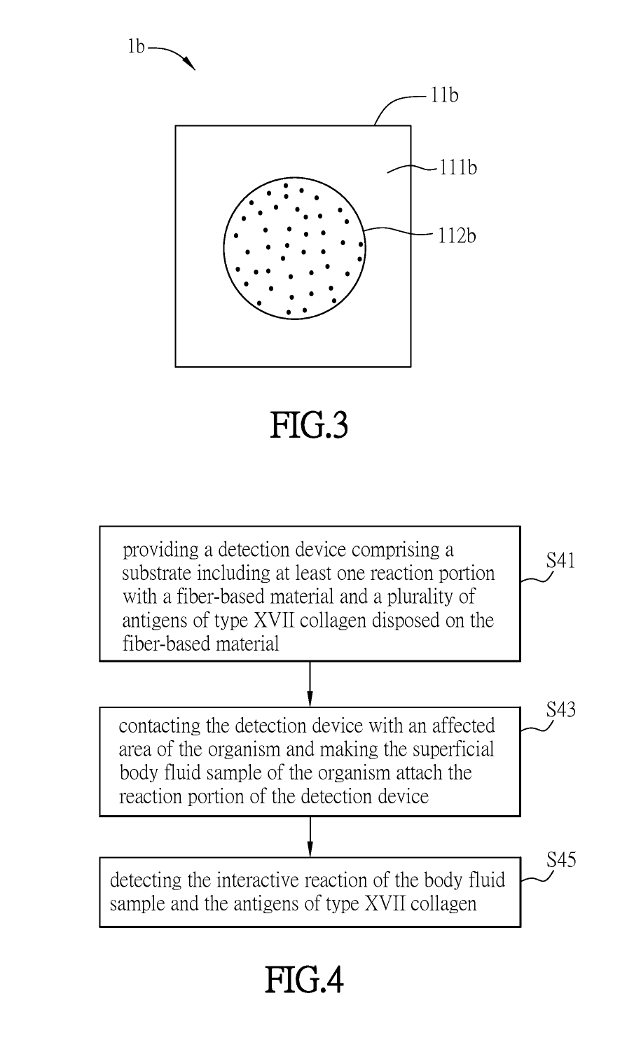

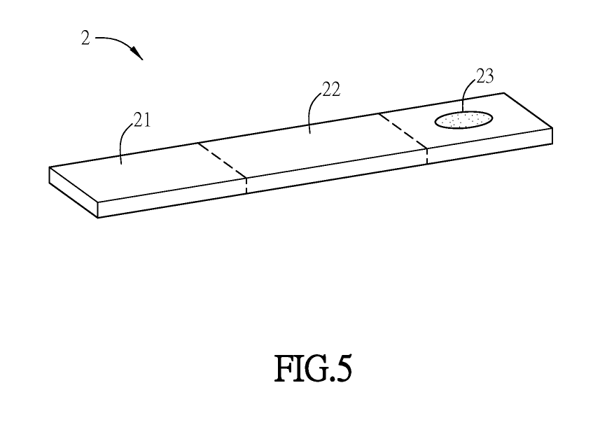

The amount of the reaction portion is not limited. In other embodiment, as shown in FIG. 3, the substrate 11b of the detection device 1b may includes only one reaction portion 112b and one non-reaction portion 111b, which designed according to the practical detection use.

With reference to FIG. 1B, in this embodiment, the detection device 1 is applied for diagnosing bullous pemphigoid. Because the patients of bullous pemphigoid may produce autoantibodies targeting the non-collagenous 16A (NC16A) domain of type XVII collagen, the detection device 1 of the present invention applies the type XVII collagen C as the detection target. However, the detailed method of disposing the antibody on the substrate is well-understood by the person having ordinary skill in the art. For example, solution including type XVII collagen C is rinsed in the reaction portion 112, then being dried in order to fix the type XVII collagen C.

After the design of the detection device 1, the detection device 1 is further applied for detecting bullous pemphigoid. In detail, the detection device 1 is contacted with an affected area, such as the blisters of the bullous pemphigoid patients, in order to make the superficial body fluid absorbed on the reaction portion 112 of the detection device 1. The fiber-based material of the reaction portion 112 is able to absorb the superficial body fluid of the affected area. By direct contacting of the detection device and the affected area, the time for tradition invasive sampling with blood drawing and additional operating may be saved. In addition, the detection device of the present invention is advantageous for relieving the patient's pain, further increasing the willingness for patients to be detected, especially for those elderly people. And because of the easy operation of the detection device 1, the operation convenience for medical personnel may be apparently increased. The patients are able to detect on their own without the limitation of environment.

Otherwise, the techniques using blood as the detection target which needs to undergone the additional processes like preprocess and separation may lost certain amount of detection sample. Thus, larger amount of detection sample must be obtained in order to achieve accurate result. On the contrary, the present invention disposes the superficial body fluid on the detection device 1 without extra process, which is advantageous for small amount of sample.

After the sampling of the superficial body fluid, the detection of bullous pemphigoid is conducted. When the superficial body fluid includes antibodies of anti-type XVII collagen, the antigens of type XVII collagen C in the reaction portion 112 may interactively react with the antibodies, especially those antibodies capable of specifically targeting the non-collagenous 16A (NC16A) domain of type XVII collagen. The detection method applying ELISA analysis is not the limitation of the present invention. Its practical detection method is as follows, the antibodies of anti-type XVII collagen are able to specifically recognize the antigen of type XVII collagen. Specific combination may occur between the antibody and the antigen. Then, the extra and uncombined superficial body fluid is washed away. The second antibody with enzyme is added and combined with the antibody of anti-type XVII collagen. Extra and uncombined second antibody is then washed away. Enzyme substrate is then added to make the enzyme show its color and thus assess whether patients diagnosed with bullous pemphigoid or not. The colorimetric results may used for estimating the amount of the antibody of anti-type XVII collagen in order to achieve the purpose of qualitative and quantitative test.

The amount of autoimmune antibodies against NC16A antigen can be determined by colorimetric reaction, fluorescence, luminescence, radiation or other signals. Specifically speaking, ELISA uses enzymes and reagents to induce colorimetric reaction so as to display presence of the antigens or analyte. Other methods comprising fluorescence, luminescence and real-time PCR reagents generating recognizable signals can also be used. The above mentioned quantitative methods are not limitation of the present invention and could be obtained in the scope of the present invention.

FIG. 4 is a flow chart showing the steps of the detection method for bullous pemphigoid according to preferred embodiment of the present invention. With reference to FIG. 4, in this embodiment, the detection method for bullous pemphigoid includes the following steps: providing a detection device comprising a substrate including at least one reaction portion with a fiber-based material and a plurality of antigens of type XVII collagen disposed on the fiber-based material (S41); contacting the detection device with an affected area of the organism and making the superficial body fluid sample of the organism attach the reaction portion of the detection device (S43); and detecting the interactive reaction of the body fluid sample and the antigens of type XVII collagen (S45). But the detailed methods applying the detection device and the steps have been disclosed by the above-mentioned description, and are not repeated here.

To improve the portability and applicability of the detection device of the present invention, the detection device 1 can be applied a kind of detection strip of the present invention.

In detail, in this embodiment, the sampling portion 21, the transferring portion 22 and the reaction portion of detection strip 2 comprise materials obtaining capillary force ability in order to provide the superficial body fluid sampled by the sampling portion 21 to conduct capillary action. The material used in sampling portion 21 and transferring portion 22 is not the limitation of the present embodiment. The materials can be chosen from cotton fiber, nitrocellulose, glass fiber, or even the same high density fiber-based material as the reaction portion 23 to improve its capillary ability.

The sampling portion 21 of the detection strip 2 also directly contact an affected area of an organism to make the superficial body fluid absorbing the sampling 21 portion of the detection strip 2. The body fluid is then transferred from the transferring portion 22 to the reaction portion 23. The transferring portion 22 may further be disposed of filtering layer in order to filter the dander or dust sampled with the body fluid simultaneously and prevent the impurities from affecting the detection.

Since the reaction portion 23 of the detection strip 2 needs to be undergone qualitative or quantitative detection, the material of the reaction portion 23 is preferably chosen from high density fiber-based material with an average pore size ranged from 0.7 to 12 micrometers, preferably ranged from 1 to 10 micrometers. The practical application range and preferable application range both includes the combination of any two integers in the above mentioned range. The superficial body fluid transferred to reaction portion can be detected according to the same method as the prior embodiment, and is not repeated here.

In addition, the detection strip is applied with a monitoring device. The monitoring device detects the interactive reaction of the body fluid sample and the antigens of type XVII collagen for qualitative or quantitative detection. The specific embodiment is conducted with the quantitative method of ELISA analysis. For example, in one embodiment, if the application uses colorimetric enzyme combined with the secondary antibody, the monitoring device may be an instrument capable of receiving optical signals to detect the color reaction of the colorimetric enzyme. Other detection methods, such as the detection of fluorescence, luminescence, and radiation, are well-understood by the person having ordinary skill in the art, and are not repeated here.

The following and accompanying figures take a number of experiments for examples to describe the practical operation method and effect of the detection device and the detailed method of the detection of bullous pemphigoid using the detection device in accordance with the embodiments of the present invention.

Experiment 1: Detection of the Monoclonal Antibody for Anti-NC16A by Paper-Based Detection Device Applying ELISA System

According to the present invention, it first provides a chromatography filter paper plate. After moistening the paper plate, 0.1 μg antigens of type XVII collagen including the NC16A domain are added thereon and stand for 5 to 7 minutes. Then, bovine serum albumin (BSA) is added as blocking agents to prevent non-specific binding. After standing for 5 to 7 minutes, antibodies of anti-type XVII collagen conjugated with HRP are added to react with the antigens for 7 to 10 minutes. Next, the second blocking agent Streptavidin is added to react with reagents for 7 to 10 minutes. Finally, the chromatography filter paper plate is rinsed. Meanwhile, the solution comprising 3,3′,5,5′-tetramethylbenzidine (TMB) and H2O2 are also added thereon until dry. After capturing the images of the paper plate, the images can be analyzed for gaining information.

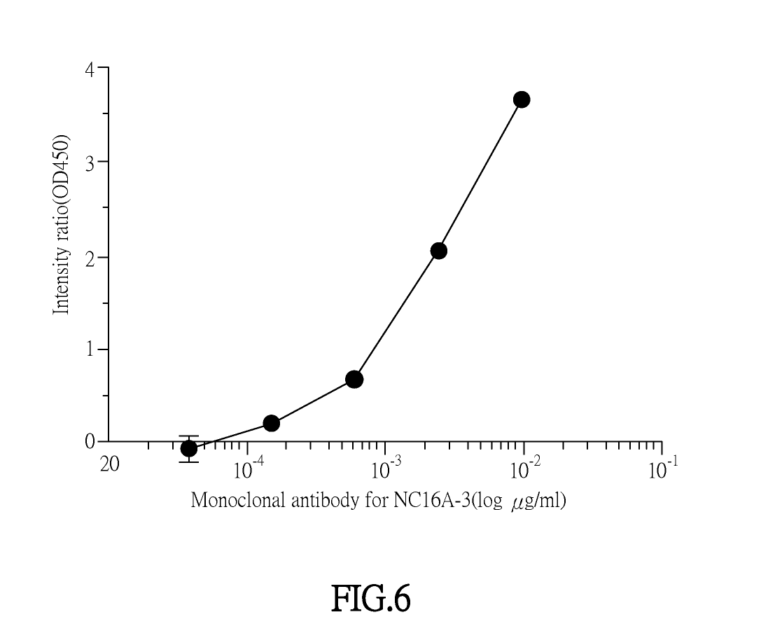

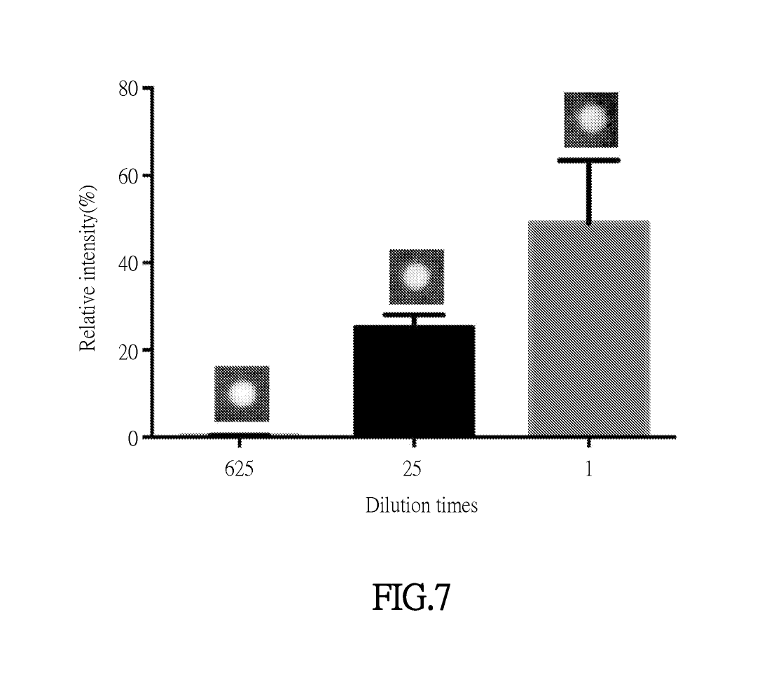

As shown in FIG. 6, they are calibration curves illustrating logarithmic value of the antibodies of anti-type XVII collagen concentration absorbed in each testing region based on average intensity of colorimetric reaction derived from HRP enzyme reacting in ELISA testing. Each point of the curve is the average value repeated for eight times (N=8) and error bars represents standard deviation of the detected results. The range of the curve between 1.6×10-1˜10-2 (log μg/mL) can be linearly approximated.

Experiment 2: the detection of Autoantibody Produced by Patients for Anti-NC16A by Paper-Based Detection Device Applying ELISA System

According to the present invention, it first provides a chromatography filter paper plate. After moistening the paper plate, 0.1 μg antigens of type XVII collagen including the NC16A domain are added thereon and stand for 5 to 7 minutes. Then, the original blister fluid of bullous pemphigoid patients is added thereon. The sample of the present invention includes 2 μl fluid without dilution, diluted 25 times, and diluted 625 times as a sample. After standing for 5 to 7 minutes, IgG antibodies which conjugate HRP are added to react with the antigens for 20 minutes. After the reaction, the sample is washed with PBST. Next, the solution comprising 3,3′,5,5′-tetramethylbenzidine (TMB) and H2O2 are also added thereon.

As shown in FIG. 7, through the reaction of the HRP enzyme in ELISA test, the average intensity of the color reaction is detected. The detection results of the three groups are compared. It is obvious that the average intensity decrease with the increase of the dilution time.

Experiment 3: the Detection of Antibody of Anti-NC16A Obtained from Patients' Serum by Paper-Based Detection Device Applying ELISA System

According to the present invention, it first provides a chromatography filter paper plate. After moistening the paper plate, 0.1 μg antigens of type XVII collagen including the NC16A domain are added thereon. Then, the serum of bullous pemphigoid patients, serum of pemphigus vulgaris, and serum of healthy person are added thereon, respectively. Next, IgG antibodies conjugated with HRP are added to react with the antigens for 20 minutes. After the reaction, the sample is washed with PBST. Next, the solution comprising 3,3′,5,5′-tetramethylbenzidine (TMB) and H2O2 are also added thereon.

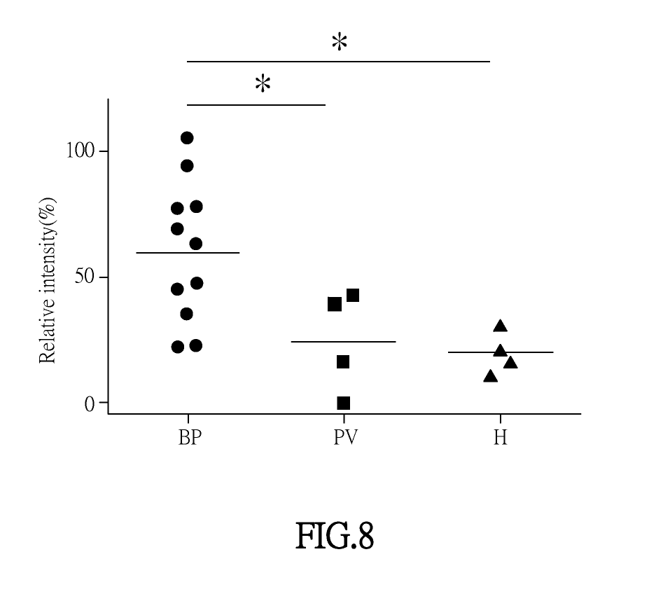

The detection result is shown as FIG. 8. The three groups including the sample obtained from serum of bullous pemphigoid patients (BP), serum of pemphigus vulgaris (PV), and serum of healthy person (H) are detected. With reference to FIG. 8, since the amount of anti-NC16A antibody is relatively high, its relative intensity distribution is apparently higher than the PV group and the H group. This also proves that the anti-NC16A antibody can be effectively applied as the detection target for bullous pemphigoid.

Experiment 4: the Detection of Anti-NC16A Antibody Obtained from Patients' Superficial Body Fluid by Paper-Based Detection Device Applying ELISA System

According to the present invention, it first provides a chromatography filter paper plate. After moistening the paper plate, 0.1 μg antigens of type XVII collagen including the NC16A domain are added thereon. Then, the superficial body fluid of bullous pemphigoid patients and blister fluid as control group are added thereon, respectively. The blister fluid in control group is obtained from the blister caused by scald, pressure ulcers or frostbite. Next, IgG antibodies which conjugate HRP are added to react with the antigens for 20 minutes. After the reaction, the sample is washed with PBST. Next, the solution comprising 3,3′,5,5′-tetramethylbenzidine (TMB) and H2O2 are also added thereon.

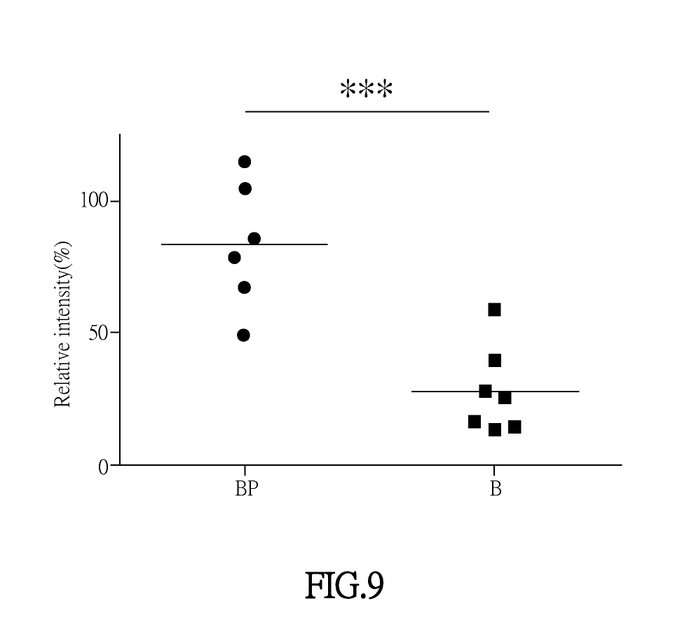

The detection result is shown as FIG. 9. The three groups including the sample obtained from superficial body fluid of bullous pemphigoid patients (BP), and superficial body fluid of scald patients (B) are detected. The so-called superficial body fluid refers to the blister fluid of the affected area of bullous pemphigoid patients and scald patients. With reference to FIG. 9, although the detection samples are both obtained from the body fluid of affected area, especially the blister affected area. Since the amount of anti-NC16A antibody is relatively high, its relative intensity distribution is apparently higher than the group B. This also proves that the anti-NC16A antibody obtained from the affected area of bullous pemphigoid patients can be effectively applied as the detection target for bullous pemphigoid.

As mentioned above, the detection device, detection method and the detection strip of the present invention are applied with a reaction portion including fiber-based material and antigens of type XVII collagen disposed thereon. The detection, detection method and the detection strip device detect the antibody included in the superficial body fluid of bullous pemphigoid patients, especially the antibody capable of specifically recognizing type XVII collagen or NC16A domain of type XVII collagen. Since the detection device, detection method and the detection strip of the present invention merely need to absorb the superficial body fluid with simple contacting or pasting onto the affected area of the patients, the time for tradition invasive sampling with blood drawing and additional operating may be saved. Preferably, the techniques using blood as the detection target which needs to undergone the additional processes like preprocess and separation may lost certain amount of detection sample. Thus, larger amount of detection sample must be obtained in order to achieve accurate result. On the contrary, the present invention disposes the superficial body fluid on the detection device without extra process, which is advantageous for small amount of sample.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

1. A detection device applied for detecting a body fluid sample, comprising:
a substrate including at least one reaction portion and at least one non-reaction portion, wherein the reaction portion is surronded and defined by the non-reaction portion, a fiber-based material is disposed on the reaction portion, and the non-reaction portion is covered by hydrophobic material; and
a plurality of antigens of type XVII collagen disposed on the fiber-based material,
wherein, the periphery of the fober-based material contacts the non-reaction portion.

2. The detection device according to claim 1, wherein the body fluid sample is a superficial body fluid sample.

3. The detection device according to claim 1, wherein the detection device is a bullous pemphigoid detection device.

4. (canceled)

5. The detection device according to claim 1, wherein the substrate includes two reaction portions, and the reaction portions are separated by the non-reaction portion.

6. The detection device according to claim 1, wherein the fiber-based material is high density fiber-based material with an average pore size ranged from 0.7 to 12 micrometers.

7. A detection method applied for detecting a superficial body fluid sample of an organism, comprising the following steps:
providing a detection device comprising a substrate including at least one reaction portion with a fiber-based material and a plurality of antigens of type XVII collagen disposed on the fiber-based material;
contacting the detection device with an affected area of the organism and making the superficial body fluid sample of the organism attach the reaction portion of the detection device; and
detecting the interactive reaction of the body fluid sample and the antigens of type XVII collagen.

8. The detection method according to claim 7, wherein the detection method is applied for detecting bullous pemphigoid.

9. The detection method according to claim 7, wherein the superficial body fluid sample includes an antibody or an antibody fragment capable of specifically recognizing the antigens of type XVII collagen.

10. A detection strip applied for detecting a body fluid sample, comprising:
a sampling portion;
a transferring portion; and
a reaction portion, wherein the transferring portion is disposed between the sampling portion and the reaction portion, the reaction portion includes a fiber-based material, and a plurality of antigens of type XVII collagen are disposed on the fiber-based material.

11. The detection strip according to claim 10, wherein the body fluid sample is a superficial body fluid sample.

12. The detection strip according to claim 10, wherein the detection strip is a bullous pemphigoid detection device.

13. The detection strip according to claim 10, wherein the fiber-based material is high density fiber-based material with an average pore size ranged from 0.7 to 12 micrometers.

14. The detection strip according to claim 10, wherein the detection strip is applied with a monitoring device, and the monitoring device detects the interactive reaction of the body fluid sample and the antigens of type XVII collagen.
